(19) United States
(12) Patent Application Publication
(10) Pub. No.: US 2014/0315262 A1
(43) Pub. Date: Oct. 23, 2014

(54) PROCESS FOR MAKING HMF AND HMF DERIVATIVES FROM SUGARS, WITH RECOVERY OF UNREACTED SUGARS SUITABLE FOR DIRECT FERMENTATION TO ETHANOL

(71) Applicant: ARCHER DANIELS MIDLAND COMPANY, Decatur, IL (US)

(72) Inventors: Alexandra Sanborn, Lincoln, IL (US); Thomas Binder, Decatur, IL (US); April Hoffart, Decatur, IL (US)

(73) Assignee: ARCHER DANIELS MIDLAND COMPANY, Decatur, IL (US)

(21) Appl. No.: 14/358,363

(22) PCT Filed: Nov. 28, 2012
(86) PCT No.: PCT/US2012/066708

§ 371 Date: May 15, 2014

Related U.S. Application Data
(60) Provisional application No. 61/584,900, filed on Jan. 10, 2012.

(51) Int. Cl. C07D 307/46; C07C 29/14; C12P 7/56; C12P 7/06; C12P 13/08

(52) U.S. Cl. CPC C07D 307/46; C12P 7/06; C12P 13/08; C12P 7/56; C07C 29/14
USPC 435/115; 549/488; 435/161; 435/139; 568/863

(57) ABSTRACT

Hydroxymethylfurfural is made from an aqueous hexose sugar solution, especially from a high fructose corn syrup product. By rapidly heating the sugar solution to the elevated temperatures involved as well as rapidly cooling the resultant product mixture, a limited per-pass conversion to HMF is obtained; correspondingly, however, the overall exposure of the HMF that is formed to acidic, elevated temperature conditions is also limited, so that byproducts are reduced. Separation and recovery of the products is simplified, and levels of HMF and other hexose dehydration products known to inhibit ethanol production by fermentation are reduced in the residual sugars product, to an extent whereby the residual sugars product is suited to be directly fermented to ethanol or for other uses.

The present invention is concerned with processes for making hydroxymethylfurfural and derivatives thereof from sugars, and particularly but without limitation, from hexose carbohydrates such as glucose and fructose.

A major product in the acid-catalyzed dehydration of fructose is 2-hydroxymethyl-5-furfuraldehyde, also known as hydroxymethylfurfural (HMF). The structure of HMF is shown below:

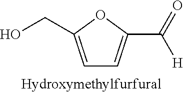

HMF represents one key intermediate substance readily accessible from renewable resources like carbohydrates, and HMF and certain derivatives of HMF (such as the ester and ether derivatives of HMF) have been proposed as biobased feedstocks for the formation of various furan monomers which are used for the preparation of non-petroleum-derived polymeric materials. While not being bound by theory, it is generally believed that fructose is converted to HMF via an acyclic pathway, although evidence also exists for the conversion to HMF via cyclic fructofuransyl intermediate pathways. Regardless of the mechanism of HMF formation, it is well known that the intermediate species formed during the reaction may in turn undergo further reactions such as condensation, rehydration, reversion and other rearrangements, resulting in a plethora of unwanted side products.

Below is one proposed pathway for the conversion of fructose to HMF:

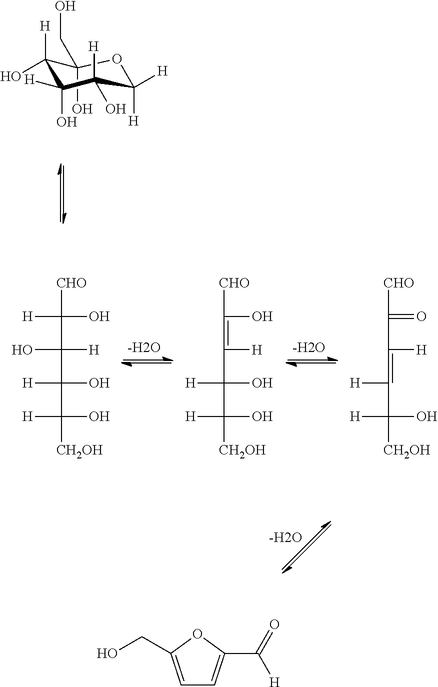

As mentioned, HMF and its related 2,5-disubstituted furanic derivatives have been viewed as having great potential for use in the field of intermediate chemicals from regrowing resources. More particularly, due to its various functionalities, it has been proposed that HMF could be utilized to produce a wide range of products such as polymers, solvents, surfactants, pharmaceuticals, and plant protection agents, and HMF has been reported to have antibacterial and anticorrosive properties. HMF is also a key component, as either a starting material or intermediate, in the synthesis of a wide variety of compounds, such as furfuryl dialcohols, dialdehydes, esters, ethers, halides and carboxylic acids.

In addition, HMF has been considered as useful for the development of biofuels, fuels derived from biomass as a sustainable alternative to fossil fuels. HMF has additionally been evaluated as a treatment for sickle cell anemia. In short, HMF is an important chemical compound and a method of synthesis on a large scale to produce HMF absent significant amounts of impurities, side products and remaining starting material has been sought for nearly a century.

Unfortunately, although it has long been known that HMF could be prepared from readily obtainable hexose carbohydrates, for example by dehydration methods, a method which provides HMF with good selectivity and in high yields has yet to be found. Complications arise from the rehydration of HMF, which yields by-products, such as, levulinic and formic acids. Another unwanted side reaction includes the polymerization of HMF and/or fructose resulting in humin polymers, which are solid waste products. Further complications may arise as a result of solvent selection. Water is easy to dispose of and dissolves fructose, but unfortunately, low selectivity and increased formation of polymers and humin increases under aqueous conditions.

Agricultural raw materials such as starch, cellulose, sucrose or inulin are inexpensive starting materials for the manufacture of hexoses, such as glucose and fructose. As shown above, these hexoses can in turn, be converted to HMF. The dehydration of sugars to produce HMF is well known. HMF was initially prepared in 1895 from levulose by Dull (Chem. Ztg., 19, 216) and from sucrose by Kiermayer (Chem. Ztg., 19, 1003). However, these initial syntheses were not practical methods for producing HMF due to low conversion of the starting material to product.

Commonly used catalysts for the preparation of HMF include cheap inorganic acids such as H₂SO₄, H₃PO₄, and HCl. These acid catalysts are used in solution and are difficult to regenerate. In order to avoid the regeneration and disposal problems, solid sulfonic acid catalysts have been used. Unfortunately, the usefulness of solid acid resins is limited because of the formation of deactivating humin polymers on the surface of the resins.

The purification of HMF has also proved to be a troublesome operation. On long exposure to temperatures at which the desired product can be distilled, HMF and impurities associated with the synthetic mixture tend to form tarry degradation products. Because of this heat instability, a falling film vacuum still must be used. Even in such an apparatus, resinous solids form on the heating surface causing a stalling in the rotor and frequent shut down time making the operation inefficient. Prior work has been performed with distillation and the addition of a non-volatile solvent like PEG-600 to prevent the buildup of solid humin polymers (Cope, U.S. Pat. No. 2,917,520). Unfortunately, the use of polyglycols leads to the formation of HMF-PEG ethers.

The prior art processes also fail to provide a method for producing HMF that can be performed economically. For example, Besemer et al Netherlands Organ. Appl. Sci. Res. Nutr. Food Res., describes the enzymatic synthesis of HMF esters. This process requires the use of expensive enzymes and therefore does not provide an economically feasible route to synthesizing HMF esters.

Garber et al., Canadian Patent 6 54240, describe the synthesis of the 2,5-tetrahydrofurandimethanol monoesters from HMF using excess amounts of anhydride and pyridine solvent. Reduction is performed using Raney Ni catalyst in diethyl ether. However the reference does not disclose the synthesis of HMF esters from fructose or using a carboxylic acid. Furthermore, the removal of Raney Ni catalyst is dangerous and the costs of disposing the catalyst may be burdensome.

In WO 2009/076627 by Sanborn et al., a method is provided of producing substantially pure HMF and HMF esters from a carbohydrate source by contacting the carbohydrate source with a solid phase catalyst; “substantially pure” was defined as referencing a purity of HMF of about 70% or greater, optionally about 80% or greater, or about 90% or greater.

A method of producing HMF esters from a carbohydrate source and organic acids involved, in one embodiment, heating a carbohydrate starting material with a solvent in a column, and continuously flowing the heated carbohydrate and solvent through a solid phase catalyst in the presence of an organic acid to form a HMF ester. The solvent is removed by rotary evaporation to provide a substantially pure HMF ester. In another embodiment, a carbohydrate is heated with the organic acid and a solid catalyst in a solution to form an HMF ester. The resulting HMF ester may then be purified by filtration, evaporation, extraction, and distillation or any combination thereof.

In WO 2009/012445 by Dignan et al., HMF is proposed to be made by mixing or agitating an aqueous solution of fructose and inorganic acid catalyst with a water immiscible organic solvent to form an emulsion of the aqueous and organic phases, then heating the emulsion in a flow-through reactor at elevated pressures and allowing the aqueous and organic phases to phase separate. HMF is present in the aqueous and organic phases in about equal amounts, and is removed from both, for example, by vacuum evaporation and vacuum distillation from the organic phase and by passing the aqueous phase through an ion-exchange resin. Residual fructose stays with the aqueous phase. High fructose levels are advocated for the initial aqueous phase, to use relatively smaller amounts of solvent in relation to the amount of fructose reacted.

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

With this in mind, the present invention relates in one aspect to a process for making HMF from an aqueous hexose sugar solution, wherein the aqueous hexose sugar solution is subjected to an acid-catalyzed dehydration to produce a mixture of HMF and unconverted sugars, then the HMF and sugars are separated by adsorption, solvent extraction or a combination of these, and the sugars are recovered in a form and condition suitable for being supplied directly to a fermentation process for producing ethanol (“fermentation-ready sugars”)—though it will be understood that for purposes of the present invention these fermentation-ready sugars need not be put to that or any other particular alternative use that might be considered, for example, in fermentations to produce lysine or lactic acid, for making levulinic acid (for example, according to a process described in a copending, commonly-assigned US patent application referenced below), for making sugar alcohols and derivative products therefrom, for making additional HMF and/or HMF derivatives by recycling to the inventive process, and so forth and so on.

In another aspect, HMF ether derivatives such as generally described in WO 2006/063220 to Sanborn can be made by the same technique and with the same benefits, through including an alcohol with the aqueous hexose solution.

In preferred embodiments according to either aspect, the aqueous hexose solution comprises one or both of glucose and fructose (more preferably being comprised of both, in the common ratios associated with commercial high fructose corn syrup products), and the acid-catalyzed dehydration step is conducted with rapid heating of the aqueous hexose solution from an ambient to a reaction temperature, as well as with rapid cooling of the HMF and/or HMF derivative unconverted sugar mixture prior to the separation of the fermentation-ready residual sugars product from the HMF and/or HMF derivative product. In addition, the time between when the aqueous hexose solution has been introduced into a reactor and the HMF and/or HMF ether products begin to be cooled is preferably limited.

By accepting limited per-pass conversion to HMF, the overall exposure of the HMF that is formed from any given aqueous hexose solution to acidic, elevated temperature conditions is limited, and preferably little to no unwanted or unusable byproducts such as humins are produced requiring waste treatments. Separation and recovery of the products is simplified and levels of HMF and other hexose dehydration products known to inhibit ethanol production by fermentation are reduced in the residual sugars product to an extent whereby the residual sugars product can be used directly for ethanol fermentation if desired. We have found, further, that processes conducted as described in greater detail below can be characterized by very high sugar accountabilities and high conversion efficiencies, with very low losses of sugars being apparent.

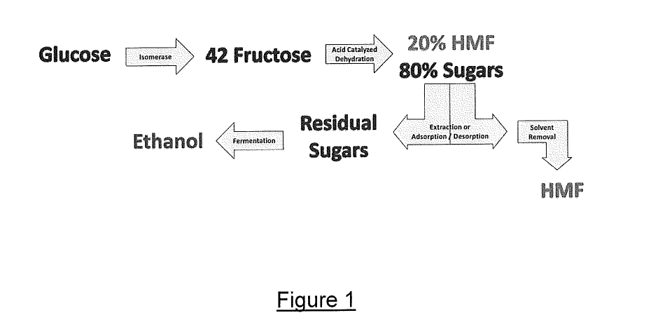

FIG. 1 is a schematic representation of a process according to the present invention in a preferred embodiment.

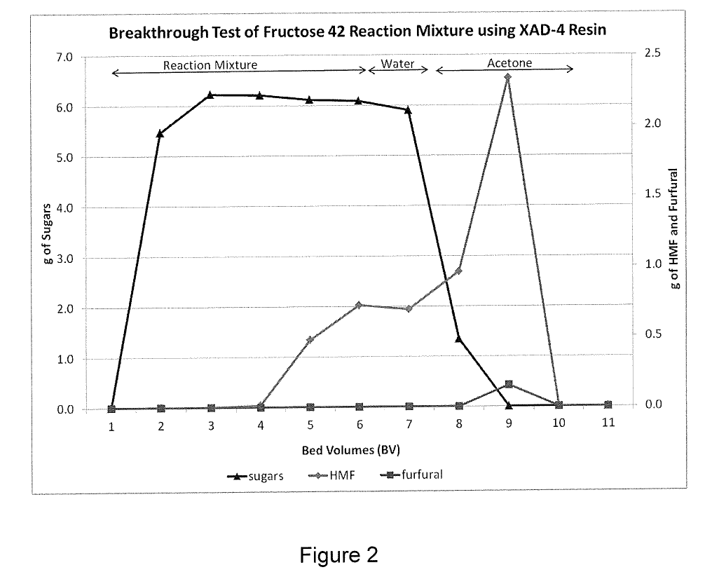

FIG. 2 depicts the results of a breakthrough test using a non-functionalized resin for separation and recovery of a residual sugars product according to one example of a process according to the present invention.

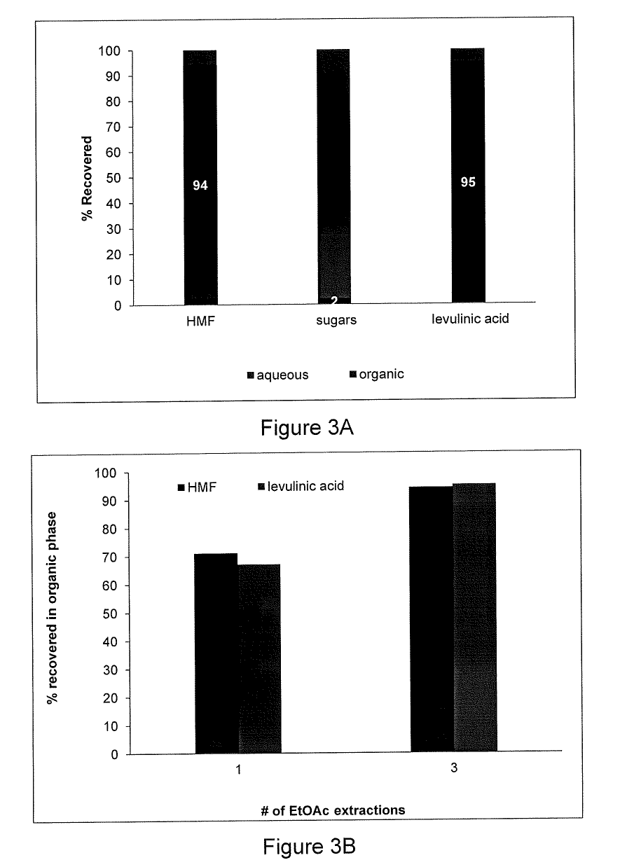

FIGS. 3A and 3B, respectively, depict the results of a separation and recovery of a residual sugars stream by solvent extraction and a breakdown of the distribution of products between the aqueous and organic phases using the solvent in question.

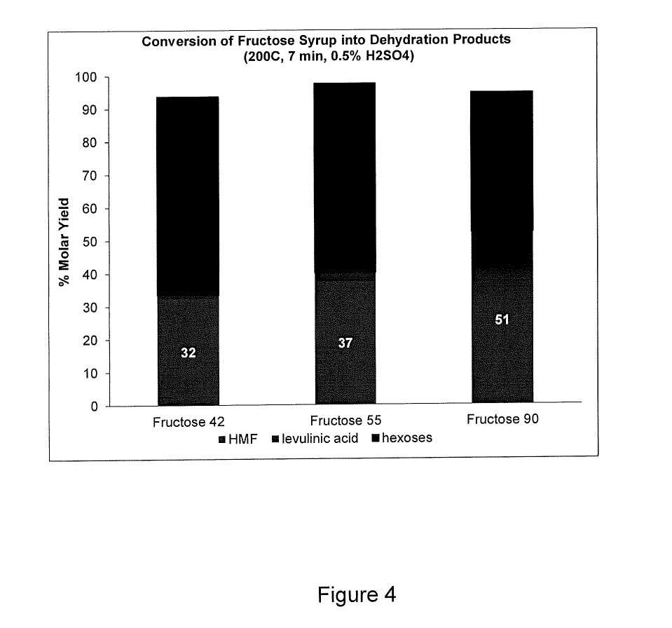

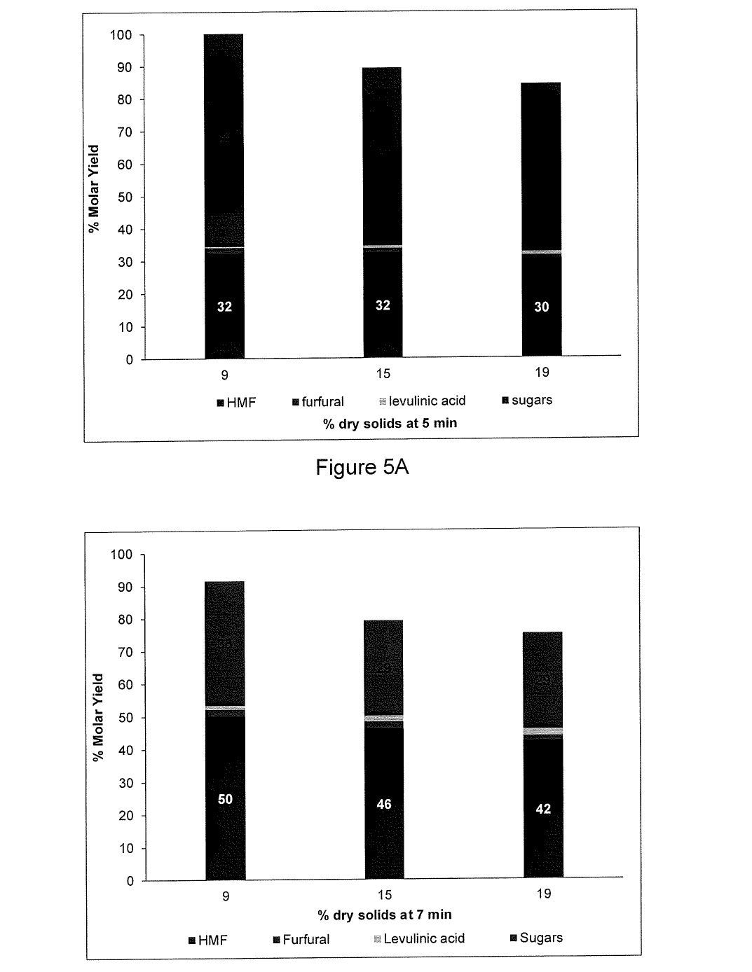

FIG. 4 depicts the product distribution differences between high fructose corn syrup products HFCS 42, HFCS 55 and HFCS 90 when identically processed in one example of a process according to the present invention.

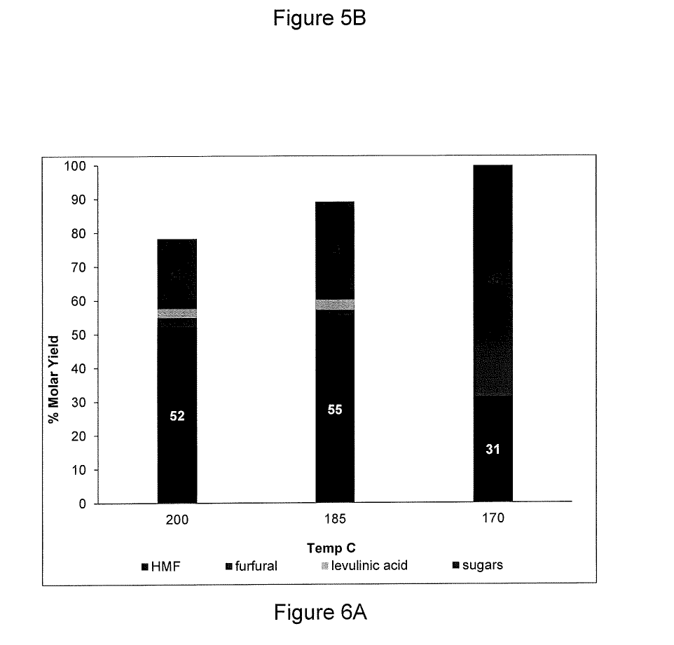

FIGS. 5A and 5B depict the sugar accountabilities and product yields resulting from processing three HFCS 90 solutions of differing concentrations, and at two different reaction times.

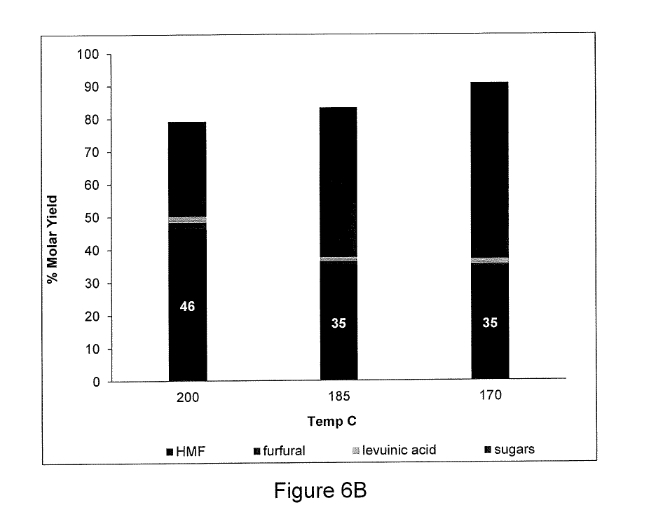

FIGS. 6A and 6B depict the effects of reaction temperature on product yield and selectivity of a single HFCS 90 solution at between 9 and 15% dissolved solids and at reaction times of 10 min and 7 min, respectively.

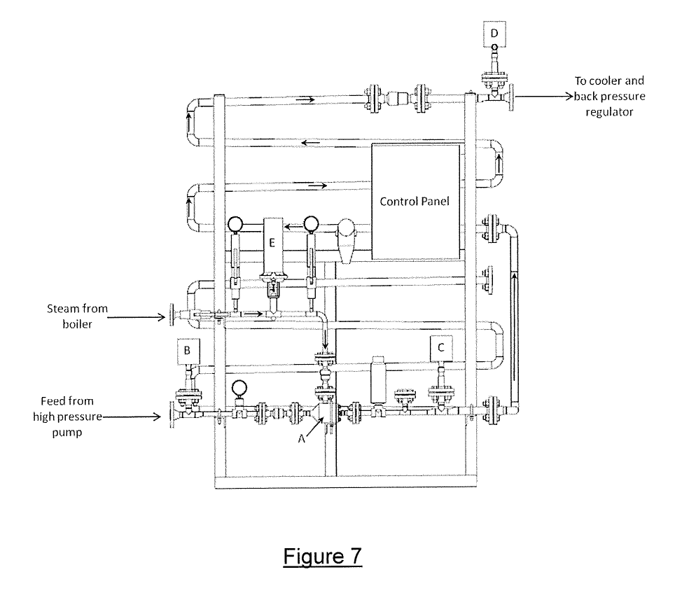

FIG. 7 shows a larger scale reactor set-up used for Examples 67-94 below.

One embodiment 10 of a process according to the present invention is shown schematically in FIG. 1. Generally, the aqueous hexose solution used can comprise one or more of the six-carbon sugars (hexoses). In particular embodiments, the aqueous hexose solution can comprise one or both of the more common hexoses glucose and fructose and in certain embodiments will comprise both of glucose and fructose. The embodiment 10 schematically shown in FIG. 1 is based on an aqueous hexose solution including both of glucose and fructose.

In the process 10, glucose as may be derived from the hydrolysis of starch with acids or enzymes or from the hydrolysis of cellulosic materials is first enzymatically converted in step 12 through use of an isomerase to a mixture of glucose and fructose, in the form of aqueous hexose sugar solution 14. Processes for making glucose from starch and for converting a portion of the glucose to fructose are well known, for example, in the making of high fructose corn syrups. Alternatively, of course, fructose derived from cane sugar or sugar beets, rather than from an isomerization of glucose, may be combined with glucose in a desired proportion. In still another embodiment, a combination of isomerization of glucose plus blending in of fructose from other known sources may be employed, to provide a combination of glucose and fructose for forming an aqueous hexose sugar solution for further processing. Conveniently, the aqueous hexose sugar solution 14 can correspond to a current high fructose corn syrup product, for example, HFCS 42 (containing about 42 percent fructose and about 53 percent glucose), HFCS 90 (made from HFCS 42 by additional purification, about 90 percent fructose and about 5 percent each of glucose and maltose) or HFCS 55 (containing about 55 percent fructose, conventionally made from blending HFCS 42 and HFCS 90), so that existing HFCS production capacity can be utilized to make HMF and derivative products to improve asset utilization and improve returns on capital, as HFCS demand and pricing and HMF and HMF derivative demand and pricing would indicate.

The aqueous hexose sugar solution 14 then undergoes an acid dehydration in step 16, to provide a mixture 18 of HMF and unconverted sugars. Because fructose dehydrates much more readily than glucose, the proportion of glucose in the mixture 18 will be higher than in the hexose sugar solution 14. The relative amounts of HMF and of the unconverted hexose sugars in the mixture 18, and the relative amounts of glucose and fructose in the unconverted sugars portion, can vary dependent on the manner in which the acid dehydration step 16 is conducted as well as on the composition of the aqueous hexose sugar solution 14. In general, of course, where HMF production is to be favored over the production of ethanol from the unconverted, residual sugars, HFCS 90 will produce more HMF given the same acid dehydration conditions than will HFCS 55, and HFCS 55 will produce more than HFCS 42 (since fructose more readily dehydrates to HMF than does glucose).

In certain embodiments, as mentioned above, the acid-catalyzed dehydration step 16 is conducted with rapid heating of the aqueous hexose sugar solution 14 from an ambient temperature to the desired dehydration reaction temperature, and then with rapid cooling of the HMF/unconverted sugar mixture 18 prior to the separation of the fermentation-ready residual sugars product from the HMF product. As well, the time from the introduction of sugar solution 14 until HMF/unconverted sugar mixture begins to be cooled is also limited.

By accepting limited per-pass conversion to HMF in this fashion, the overall exposure of the HMF that is formed to acidic, elevated temperature conditions is correspondingly limited, so that preferably little to no unwanted or unusable byproducts such as humins are produced requiring waste treatments. Separation and recovery of the products is simplified and levels of HMF and other hexose dehydration products known to inhibit ethanol production by fermentation are reduced in the residual sugars product to an extent whereby the residual sugars product can be used directly for ethanol fermentation if desired.

Consequently, typically the mixture 18 will comprise from 10 to 55 percent molar yield of HMF, from 30 to 80 percent molar yield of unconverted, residual sugars, and not more than 10 percent molar yield of other materials such as furfural, levulinic acid, humins etc. Preferably, the mixture 18 will comprise from 30 to 55 percent yield of HMF, from 40 to 70 percent yield of unconverted, residual sugars, and not more than 5 percent yield of other materials such as furfural, levulinic acid, humins etc. More preferably, the mixture 18 will comprise from 45 to 55 percent yield of HMF, from 25 to 40 percent yield of unconverted, residual sugars, and not more than 5 percent yield of other materials such as furfural, levulinic acid, humins etc.

Returning now to FIG. 1, the HMF and unconverted, residual sugars in mixture 18 are then separated by adsorption, solvent extraction, or a combination of these in separation step 20, to yield an HMF product stream or portion 22 and a fermentation-ready sugars stream or portion 24 which can optionally be supplied to an ethanol fermentation step 26 for producing an ethanol product 28.

Adsorption in step 20 can be by means of any material which preferentially adsorbs HMF from the residual hexose sugars in the mixture 18. A material which has been found to be very effective at retaining the HMF and the small amounts of levulinic acid formed is DOWEX® OPTIPORE® V-493 macroporous styrene-divinylbenzene resin (CAS 69011-14-9, The Dow Chemical Company, Midland, Mich.), which has been described by its manufacturer as having a 20-50 mesh particle size, a 46 angstrom mean pore size and 1.16 mL/g pore volume, a surface area of 1100 sq. meters/g and a bulk density of 680 g/liter. An ethanol wash was effective for desorbing most of the adsorbed HMF, and subsequent washing of the resin with acetone provided quantitative recovery of the HMF that was adsorbed. An alternative is AMBERLITE™ XAD™-4 polystyrene divinylbenzene polymeric adsorbent resin (CAS 37380-42-0, Rohm & Haas Company, Philadelphia, Pa.), a non-functionalized resin having a 1.08 g/mL dry density, a surface area of 725 square meters per gram, an average pore diameter of 50 angstroms, a wet mesh size of 20-60 and a pore volume of 0.98 mL/gram. Other suitable adsorbents can be activated carbon, zeolites, alumina, clays, non-functionalized resins (LEWATIT® AF-5, LEWATIT® S7968, LEWATIT® VPOC1064 resins, all from Lanxess AG), Amberlite® XAD-4 macroreticular crosslinked polystryrene divinylbenzene polymer resin (CAS 37380-42-0, Rohm & Haas Company, Philadelphia, Pa.), and cation exchange resins, see U.S. Pat. No. 7,317,116 B2 (Sanborn) and the later U.S. Pat. No. 7,897,794 (Geier and Soper). Desorption solvents may include polar organic solvents, for example, alcohols such as ethanol, amyl alcohol, butanol and isopentyl alcohol, as well as ethyl acetate, methyl tetrahydrofuran and tetrahydrofuran.

Suitable solvents for solvent extraction include methyl ethyl ketone and especially ethyl acetate, due to the latter's great affinity for HMF and levulinic acid, low boiling point (77 deg. C.) and ease of separation from water. As demonstrated in certain of the examples below, virtually complete recovery of the sugars and of the HMF from mixture 18 was accomplished through a series of ethyl acetate extractions. Additionally, while the residual sugars recovered by other means were still suitable for being directly processed to ethanol in the subsequent ethanol fermentation step 26, those recovered following the quantitative extraction with ethyl acetate were observed to be significantly less inhibitory even under non-optimal conditions. A variety of other solvents have been suggested or used in the literature related to HMF and HMF derivative synthesis and recovery in biphasic systems, and these may be appropriate for use in the context of the present invention. Examples of other useful solvents are butanol, isoamyl alcohol, methyl ethyl ketone, methyl isobutyl ketone, diethyl ether, cyclopentyl dimethyl ether, methyl tetrahydrofuran, and methyl butyl ether.

Ethanol fermentation step 26 can encompass any known process whereby a hexose sugars feed of the type represented by fermentation-ready sugars stream or portion 24 may be converted to one or more products inclusive of ethanol, at least in some part by fermentation means. Both aerobic and anaerobic processes are thus contemplated, using any of the variety of yeasts (e.g., kluyveromyces lactis, kluyveromyces lipolytica, saccharomyces cerevisiae, s. uvarum, s. monacensis, s. pastorianus, s. bayanus, s. ellipsoidues, candida shehata, c. melibiosica, c. intermedia) or any of the variety of bacteria (e.g., clostridium sporogenes, c. indolis, c. sphenoides, c. sordelli, candida bracarensis, candida dubliniensis, zymomonas mobilis, z. pomaceas) that have ethanol-producing capability from the fermentation-ready sugars stream or portion 24 under aerobic or anaerobic conditions and other appropriate conditions. The particular yeasts (or bacteria) used and other particulars of the fermentations employing these various yeasts (or bacteria) are a matter for routine selection by those skilled in the fermentation art, though the examples below demonstrate the functionality of one common anaerobic yeast strain, saccharomyces cerevisiae. Given that the sugars stream or portion 24 derives from a process for making the acid dehydration product HMF, a yeast or bacteria that has been demonstrated for use particularly with sugars derived from a lignocellulosic biomass through acid-hydrolyzing the biomass and/or a cellulosic fraction from biomass may be preferred. For example, the aerobic bacterium corynebacterium glutamicum R was evaluated in Sakai et al., “Effect of Lignocellulose-Derived Inhibitors on Growth of and Ethanol Production by Growth-Arrested Corynebacterium glutamicum R”, Applied and Environmental Biology, vol. 73, no. 7, pp 2349-2353 (April 2007), as an alternative to detoxification measures against organic acids, furans and phenols byproducts from the dilute acid pretreatment of biomass, and found promising.

While the amounts of HMF (and/or HMF ethers, as the case may be) and of unconverted, residual sugars may vary somewhat, preferably in all embodiments a high degree of sugar accountability is achieved, where “sugar accountability” is understood to refer to the percentage of sugars input to the acid dehydration step 16 that can be accounted for in adding the molar yields of identifiable products in the mixture 18—essentially adding the molar yields of HMF (and/or of HMF ethers), levulinic acid, furfural and residual, unconverted sugars. Preferably, a process according to the present invention is characterized by a total sugar accountability of at least 70 percent, more preferably at least 80 percent and most preferably at least 90 percent.

The fermentation-ready sugars stream or portion 24 can, in whole or in part, also be used for other purposes beyond the production of ethanol. For example, sugars in stream or portion 24 can be recycled to the beginning of the acid dehydration step 16 for producing additional HMF or HMF ethers. The hexose sugars represented by stream or portion 24 can also be hydrogenated to sugar alcohols for producing other biobased fuels and fuel additives (other than or in addition to ethanol), see, for example, U.S. Pat. No. 7,678,950 to Yao et al. The sugars in stream or portion 24 can be fermented to produce lysine or lactic acid according to known methods, or used for making another dehydration product such as levulinic acid. Still other uses will be evident to those skilled in the art, given the character of the sugars stream or portion 24 provided by the described process.

A number of prospective uses of HMF product stream or portion 22 have already been mentioned, but one important contemplated use would be in the manufacture of 2,5-furandicarboxylic acid (FDCA) using a Mid-Century type Co/Mn/Br oxidation catalyst under oxidation conditions, as described in United States Pat. Application Publication No. US 2009/1056841 to Sanborn et al. and in copending Patent Cooperation Treaty Application Ser. No. PCT/US12/52641, filed Aug. 28, 2012 for “Process for Producing Both Biobased Succinic Acid and 2,5-Furandicarboxylic Acid”, both of which are now incorporated herein by reference. Another contemplated use would be for making the more thermally-stable intermediate levulinic acid, particularly according to copending and commonly-assigned U.S. Patent Application Ser. No. 61/584,890, filed Jan. 10, 2012, for “Process for Making Levulinic Acid”, which application is also incorporated by reference herein.

The acid dehydration step 16 is preferably conducted in a manner to limit per-pass conversion to HMF and the exposure of the HMF that is formed to acidic, elevated temperature conditions. Rapid heating of the hexose sugar solution 14, as well as rapid cooling of the HMF/unconverted sugar mixture produced from the acid dehydration step 16, are desirable for accomplishing these objectives for a given amount of hexose sugar solution 14. Further, once the aqueous hexose solution 14 has reached the desired reaction temperature range, the extent to which the aqueous hexose solution remains subject to the acidic, elevated temperature conditions is preferably also limited. While optimal conditions will vary somewhat from one embodiment to the next, for example, in processing HFCS 42 versus HFCS 55 versus HFCS 90 as shown clearly below, in general terms for a concentrated sulfuric acid content of about 0.5 percent by weight based on the mass of hexose sugars in the sugar solution 14 (or the equivalent acid strength, for other acid catalysts), a reaction temperature of from 175 degrees Celsius to 205 degrees Celsius, a dry solids loading of sugars in the range of from 10 to 50 percent, a final dry solids concentration of from 10 to 25 percent, and an average residence or reaction time of from 2 to 10 minutes appear to be advantageous. “Average residence or reaction time” or similar terminology as used herein refers to the time elapsed from the introduction of the sugar solution 14 into a reactor until cooling of the mixture 18 is commenced.

As a general matter, of course, it would be preferable to process sugar solutions 14 having a greater loading of the hexose sugars rather than a lesser loading, though some trade-offs were observed in terms of overall sugars accountability and in other respects, and these would need to be considered in determining the optimum conditions to be observed for a given feedstock. Similarly, milder reaction conditions generally provide lesser conversion, but enable increased sugars accountability.

For the particular example of a 40 percent dry solids loading HFCS 42 feed providing up to a 20 percent final dry solids concentration, using a shorter reaction time and a temperature toward the higher end seem preferable, for example, 5 minutes at 200 degrees Celsius. For HFCS 90, given the same acid starting concentration, the reaction temperature can be in the range of from 185 degrees to 205 degrees Celsius, the dry solids loading of hexose sugars in the sugar solution 14 can be from 30 to 50 percent and provide an 8 to 15 percent final dry solids concentration, and a reaction time can be from 5 to 10 minutes.

As an illustration of the considerations involved in processing one feedstock versus another, for HFCS 90 in contrast to HFCS 42, a final dry solids concentration of 20 percent could not be processed with the same overall sugars accountability, and a lower final dry solids concentration was indicated as preferable. For a final dry solids concentration of 10 percent, a reaction temperature of 185 degrees Celsius and a reaction time of 10 minutes were observed to provide favorable results. Favored conditions for the recovered sugars in stream or portion 24, it should be noted, may differ from those contemplated for freshly-supplied sugars in sugar solution 14 where recycle is contemplated for making additional HMF product or levulinic acid.

In any event, the heating to the desired reaction temperature is preferably accomplished in not more than 15 minutes, preferably is accomplished in 11 minutes of less, more preferably in not more than 8 minutes and still more preferably is accomplished in not more than five minutes. As demonstrated by the examples given hereafter, rapid feeding of a quantity of ambient hexose sugar solution to a hot aqueous acid matrix (in two minutes) gave consistent improvements in one or more of HMF selectivity, yield and overall sugar accountability compared to less rapid feeding, even given the same elapsed time between when the quantity of hexose sugar solution was fully introduced and when cooling was initiated. Rapid cooling from the reaction temperature to 50 degrees Celsius and lower is preferably accomplished in not more than 5 minutes, especially 3 minutes or less.

More particularly, in a batch reactor (as clearly shown in the examples below) combining the sugar solution 14 and the acid catalyst in a hot reactor already close to or at the desired reaction temperature provides improved results as compared to where the sugar solution 14 and acid catalyst are added to a reactor and then heated gradually together to the desired reaction temperature.

In regard to continuous processes, one suitable means for rapidly heating the sugar solution 14 and the acid catalyst would be direct steam injection. A commercially-available, in-line direct steam injection device, the Hydro-Thermal Hydroheater™ from Hydro-Thermal Corporation, 400 Pilot Court, Waukesha, Wis., injects sonic velocity steam into a thin layer of a liquid (such as the sugar solution 14) flowing from an inlet pipe through a series of gaps. Steam flow is adjusted precisely through a variable area nozzle to an extent whereby outlet fluid temperatures are claimed to be controllable within 0.5 degrees Fahrenheit over a large liquid turndown ratio. Turbulent mixing takes place in a specifically designed combining tube, with an adjustable degree of shear responsive to adjustments of the steam flow and the liquid flow through (or pressure drop across) the series of gaps. Devices of this general character are described in, for example, U.S. Pat. Nos. 5,622,655; 5,842,497; 6,082,712; and 7,152,851.

In The examples reported below using such a device, in a reaction system shown in FIG. 7, the highest HMF yield and sugar accountability from HFCS 42 syrup included a system of sulfuric acid (0.5% by wt of sugars), an initial dry solids concentration of 20% and rapid heating of the reaction mixture by direct steam injection by means of a Hydro-Thermal Hydroheater™ (at A) with a system back pressure of 215-220 psig, a steam pressure of 275 psig, a time of 5-6 minutes at the reaction temperatures provided by the direct steam injection and rapid cooling of the product mixture before pressure relief. The reaction control set point, as monitored by the temperature control element (C), was 200 degrees C. and the maximum temperature achieved at the end of the resting tube (at D) was 166 degrees C. HMF was obtained with these conditions in up to 20% molar yield with greater than 90% total sugar accountability. There was virtually no visible production of insoluble humins.

For HFCS 90 syrup processed in the same apparatus, the highest HMF yield and sugar accountability included a system of sulfuric acid (0.5% by wt of sugars) an initial dry solids concentration of 10% and rapid heating of the reaction mixture by direct steam injection with a system back pressure of 150 psig, a steam pressure of 200 psig, a time of 11 minutes at the reaction temperatures provided by the direct steam injection and rapid cooling of the product mixture before pressure relief. The reaction control set point was 185 degrees C. and the maximum temperature achieved at the end of the resting tube was 179 degrees C. HMF was obtained from HFCS 90 with these conditions up to 31% molar yield with greater than 95% total sugar accountability. There was again virtually no visible production of insoluble humins.

Rapid cooling of the mixture 18 can be accomplished by various means. For example, while a brazed plate heat exchanger was used in at least certain of the examples below prior to a pressure reduction, other types of exchangers could be used. Other options will be evident to those of routine skill in the art

It will be appreciated that the acid-catalyzed dehydration step 16 can be conducted in a batchwise, semi-batch or continuous mode. A variety of acid catalysts have been described previously for the dehydration of hexose-containing materials to HMF, including both homogeneous and heterogeneous, solid acid catalysts. Solid acid catalysts would be preferred given they are more readily separated and recovered for reuse, but selecting a catalyst that will maintain a satisfactory activity and stability in the presence of water and at the temperatures required for carrying out the dehydration step 16 can be problematic. Consequently, sulfuric acid has been used in the examples which follow, and provided good yields and excellent sugar accountabilities in the inventive process.

The present invention is illustrated by the following examples:

### EXAMPLES 1-26

For Examples 1-26, an initial series of carbohydrate dehydration reactions was performed at a bench scale, using a Parr multireactor system (Parr Instrument Company, Moline, IL). For each run, a 75 mL reaction chamber was first charged with an acidic aqueous solution. The acidic aqueous solution was heated to the specified temperature over a period of 20-30 min with magnetic stirring at a controlled rate of about 850 rpm. Once the desired temperature was reached, a room temperature HFCS 42-based sugar solution was rapidly introduced into the acidic aqueous solution by an Eldex high pressure pump (Eldex Laboratories, Inc, Napa, Calif.) over a period of about 20 to 120 sec. The reaction was continued for a certain time, then the product was flowed through a cooling coil consisting of ⅛″ stainless steel tubing and into a collection vial. Analysis of the samples was by HPLC. The results are provided in Table 1 below.

TABLE 1
Experimental conditions and product yields,
HFCS 42 syrup dehydrations.

| | | | Final | | | | |
|---|---|---|---|---|---|---|---|
| | | | dry | | | | |
| | | | solids | % molar yield | | | |
| Entry | Time | Temp | in | | | levulinic | C6 |
| # | (min) | (C.) | reactor | HMF | furfural | acid | sugars |
| 1 | 2 | 193 | 4.6 | 15 | 0 | 0 | 78 |
| 2 | 5 | 199 | 4.6 | 33 | 0 | 0 | 66 |
| 3 | 10 | 201 | 4.6 | 47 | 2 | 0 | 48 |
| 4 | 15 | 199 | 4.6 | 44 | 2 | 0 | 40 |
| 5 | 2 | 204 | 9.1 | 27 | 13 | 1 | 83 |
| 6 | 5 | 214 | 9.1 | 41 | 3 | 3 | 54 |
| 7 | 5 | 220 | 4.8 | 43 | 3 | 4 | 49 |
| 8 | 10 | 214 | 5.0 | 33 | 3 | 9 | 44 |
| 9 | 5 | 214 | 9.1 | 41 | 3 | 3 | 60 |
| 10 | 10 | 215 | 9.1 | 31 | 2 | 10 | 44 |
| 11 | 15 | 215 | 9.1 | 22 | 4 | 14 | 34 |
| 12 | 2 | 197 | 9.1 | 21 | 1 | 0 | 102 |
| 13 | 5 | 201 | 9.1 | 37 | 1 | 1 | 86 |
| 14 | 10 | 199 | 9.1 | 41 | 0 | 5 | 72 |
| 15 | 15 | 200 | 9.1 | 35 | 1 | 7 | 56 |
| 16 | 5 | 203 | 5.0 | 30 | 2 | 1 | 70 |
| 17 | 10 | 199 | 4.9 | 40 | 2 | 2 | 67 |
| 18 | 2 | 189 | 8.9 | 22 | 0 | 0 | 95 |
| 19 | 5 | 200 | 9.2 | 40 | 2 | 2 | 69 |
| 20 | 10 | 201 | 9.3 | 38 | 2 | 7 | 52 |
| 21 | 15 | 200 | 9.3 | 33 | 2 | 10 | 48 |
| 22 | 2 | 198 | 15.0 | 33 | 2 | 2 | 70 |
| 23 | 5 | 196 | 14.8 | 32 | 2 | 4 | 58 |
| 24 | 7 | 211 | 14.8 | 33 | 2 | 6 | 46 |
| 25 | 10 | 200 | 15.5 | 23 | 2 | 11 | 45 |
| 26 | 5 | 198 | 20.0 | 32 | 1 | 2 | 69 |

### EXAMPLES 27-32

Based upon the results seen with the bench scale examples, a series of continuous bench scale runs were conducted with the same HFCS 42 feedstock. For these examples, a 15% dry solids solution with 0.5% sulfuric acid by the total sugars weight was passed through a heated stainless steel coil ( 1/16″ tubing, 222 cm in length) maintained at a selected temperature ranging from 185 degrees to 205 degrees Celsius, at flow-through times ranging from about 2.7 to about 4.0 minutes. The backpressure of the system was maintained at 40-70 bar through the use of a backpressure regulator obtained from Upchurch Scientific. Products were then flowed through a cooling coil (stainless steel, 1/16″ tubing), collected, and analyzed by HPLC methods, with the results shown in Table 2: No clogging of the system was observed, suggesting little formation of insoluble polymers or of humins.

TABLE 2
Conditions and product yields, continuous conversion of HFCS 42 syrup.

| | | | | | | | | selectivity | |
|---|---|---|---|---|---|---|---|---|---|
| | | | | % molar yield from sugars | | | | to | sugar |
| | time | temp | | levulinic | | | total | dehydration | conversion |
| entry # | (min)* | (C.) | HMF | acid | furfural | fructose | knowns | products | % |
| 1 | 2.78 | 185 | 2 | 0 | 0 | 101 | 104 | — | 0 |
| 2 | 2.71 | 195 | 5 | 0 | 0 | 95 | 101 | 119 | 5 |
| 3 | 2.78 | 200 | 8 | 0 | 0 | 91 | 99 | 94 | 9 |
| 4 | 3.29 | 200 | 10 | 0 | 0 | 89 | 99 | 91 | 11 |
| 5 | 3.69 | 200 | 11 | 0 | 0 | 87 | 98 | 87 | 13 |
| 6 | 4.03 | 200 | 12 | 0 | 0 | 85 | 98 | 87 | 15 |
| average | 4.00 | 205 | 16 | 0 | 0 | 78 | 94 | 78 | 22 |

*based on actual feed rate.
% selectivity = moles dehydration products/moles of sugar reacted * 100.
Conditions: 0.5% sulfuric acid by wt sugars in 15% dry solids.

### EXAMPLES 33-34

An aggregate sample of all of the products obtained from Examples 27-32—corresponding to an average retention or flow-through time of 4.00 minutes at 205 degrees Celsius—was treated with an adsorbent resin, DOWEX™ OPTIPORE™ V493 general purpose, highly cross-linked styrene-divinylbenzene macroporous resin (CAS 69011-14-9, The Dow Chemical Company, Midland, Mich.) at 30 percent by weight of resin of the whole. The combination was stirred at 40 degrees Celsius using an oil bath for 2 hours, then vacuum filtered to separate the resin and a light yellow filtrate. About 100 grams of ethanol was added to the wet resin, and the combination was again stirred using an oil bath at 35 degrees Celsius for an additional two hours before undergoing a second vacuum filtration to provide the resin and a maroon filtrate. An additional 50 mL of acetone was then added to the wet resin, the combination was stirred at room temperature for an additional two hours and then the combination was vacuum filtered a third time to provide a third filtrate sample.

The respective filtrates were then analyzed by high performance liquid chromatography, and the first filtrate was found to contain 94 percent of the total unconverted sugars remaining. About 68 percent of the HMF was adsorbed to the resin, by comparison, and about 92 percent of this was removed with an ethanol wash into the second filtrate. Subsequent washing of the resin with acetone provided a quantitative recovery of the remaining HMF that was adsorbed, in the third filtrate.

A second aggregate sample was subjected to a breakthrough test using a different, non-functionalized resin, Amberlite® XAD-4 macroreticular crosslinked polystryrene divinylbenzene polymer resin (CAS 37380-42-0, Rohm & Haas Company, Philadelphia, Pa.). The results are shown in FIG. 2, and indicate a recovery after water and acetone washes of 98 percent of the HMF in the adsorbed/desorbed HMF product, and 95 percent of the residual sugars in the residual sugars product.

### EXAMPLES 35-37

Two other aggregate samples of all of the products obtained from Examples 27-32 were separated into HMF and residual sugar products by adsorption/desorption with DOWEX™ OPTIPORE™ V493 general purpose, highly cross-linked styrene-divinylbenzene macroporous resin and with using ethanol for desorption of the adsorbed HMF (no acetone for entries 1 and 2 of Table 3), while a third aggregate sample was three-times solvent extracted with ethyl acetate (entry 3). The compositions of the recovered residual sugar products from the three samples are shown in Table 3 as follows:

TABLE 3
Chemical composition of the sugars obtained
following separation of HMF.

| | | Concentration | | | | | | |
|---|---|---|---|---|---|---|---|---|
| | | (wt %) | | | | | | |
| Entry | Purification | Glu- | Fruc- | Levo- | Other | | Fur- | Lev. |
| # | Method | cose | tose | glucosan | sugars | HMF | fural | Acid |
| 1 | Adsorption | 7.15 | 3.88 | 0.22 | 0.78 | 0.50 | 0.00 | 0.01 |
| 2 | Adsorption | 7.28 | 1.72 | nd | 0.92 | 0.75 | 0.00 | 0.26 |
| 3 | Extraction | 7.97 | 1.93 | nd | 1.24 | 0.40 | 0.00 | 0.01 |

nd = not detected.

These three sugar fractions were forwarded for fermentation with saccharomyces cerevisiae. Ethanol yields for entry #2 in Table 3 were from 77 to 80 percent. No inhibition was observed for any of the sugar fractions and viability remained constant.

### EXAMPLE 38

An aggregate product mixture from the combined products of examples 74-77 in Table 5 below was solvent-extracted with three portions of ethyl acetate, with analysis of the aqueous and organic phases following each extraction episode. FIG. 3A compares the effectiveness of one extraction and three extractions, and demonstrates that three extractions recover a high percentage of the HMF and levulinic acid dehydration products. FIG. 3B shows the distribution of HMF, residual sugars and levulinic acid products between the aqueous and organic extraction phases, and establishes that ethyl acetate very effectively separates the residual sugars and the HMF and levulinic acid dehydration products from one another.

### EXAMPLE 39

The aqueous fraction containing the residual sugars accumulated from the three ethyl acetate extractions in Example 38 was analyzed by HPLC methods, and determined to contain 10.4 percent by weight of fructose, 12.2 percent by weight of glucose, 2.5 weight percent of HMF and 0.5 weight percent of levulinic acid, by total mass. With further rapid heating to 200 degrees Celsius and holding the aqueous fraction at this temperature for various periods of time ranging from 2.5 minutes up to 12 minutes, up to 98 percent conversion of the fructose was realized after 4 to 5 minutes of reaction time while glucose conversion was much lower. Overall sugar accountabilities ranged from just over 90 percent at 2.5 minutes reaction time down to just over 70 percent for 12 minutes reaction time just with heating, whereas the addition of a further 0.65 percent of sulfuric acid brought sugar accountabilities of more than 90 percent (at 12 minutes reaction/hold time) up to 100 percent (at reaction times of 7 minutes and less). Dehydration products were produced in excess of fifty percent combined molar yield for a reaction time of at least 4.75 minutes, whereas dehydration product yield on a combined molar percent basis was in all cases not more than about 40 percent in the absence of additional acid.

### EXAMPLES 40-51

Additional portions of the products generated in Examples 27-32 were then either contacted with an adsorbent or solvent-extracted as indicated in the following Table 3, to separate out and recover a residual sugars fraction for fermentation testing in parallel bioreactors from DASGIP Biotools, LLC, Shrewsbury, Mass., using the same saccharomyces cerevisiae yeast strain but different run pH's and inoculum levels. Results are shown in Table 3, and show recovered sugars may be suitably used directly for ethanol production:

TABLE 3
Results of Fermentation Testing

| | | | | | | Ethanol | Glucose | | Fructose | |
|---|---|---|---|---|---|---|---|---|---|---|
| Purif. | % in | Run | Inoc | EFT | Productivity | Produced | Available | % | Available | |
| Method | Media | pH | Level | (hr)¹ | g/l/hr | (g) | (g/L) | Used | (g/L) | % Used |
| Carbon | 40 | 4 | 10% | 48 | 0.36 | 17.30 | 266.89 | 16.04 | 15.20 | 10.84 |
| Carbon | 40 | 4 | High | 48 | 1.77 | 84.80 | 266.89 | 71.68 | 14.80 | 29.51 |
| Carbon | 40 | 4.5 | 10% | 48 | 2.04 | 97.90 | 266.89 | 84.83 | 14.30 | 37.27 |
| Carbon | 40 | 4.5 | High | 48 | 2.40 | 115.40 | 266.89 | 97.18 | 14.90 | 58.12 |
| EtOAc | 40 | 4 | 10% | 48 | 2.63 | 126.30 | 266.45 | 99.61 | 18.20 | 100.00 |
| EtOAc | 40 | 4 | High | 48 | 2.70 | 129.70 | 266.45 | 99.55 | 19.00 | 100.00 |
| EtOAc | 40 | 4.5 | 10% | 48 | 2.51 | 120.40 | 266.45 | 99.54 | 19.10 | 100.00 |
| EtOAc | 40 | 4.5 | High | 48 | 2.65 | 127.20 | 266.45 | 99.61 | 18.90 | 100.00 |
| V493 | 40 | 4 | 10% | 48 | 1.40 | 67.20 | 263.42 | 62.42 | 12.49 | 31.44 |
| resin | | | | | | | | | | |
| V493 | 40 | 4 | High | 48 | 2.09 | 100.30 | 263.42 | 83.71 | 12.71 | 47.55 |
| resin | | | | | | | | | | |
| V493 | 40 | 4.5 | 10% | 48 | 2.45 | 117.60 | 263.42 | 99.35 | 12.35 | 82.86 |
| resin | | | | | | | | | | |
| V493 | 40 | 4.5 | High | 48 | 2.56 | 123.10 | 263.42 | 99.83 | 12.44 | 100.00 |
| resin | | | | | | | | | | |

EFT = estimated fermentation time;
C = adsorption by CENTAUR ® 12X40 bituminous coal activated carbon (Calgon Carbon Corporation, Pittsburgh, PA);
EtOAc = ethyl acetate solvent extraction;
y493 = DOWEX ™ OPTIPORE ™ V493 adsorbent

### EXAMPLES 52-54

Because glucose does not dehydrate as readily as fructose to HMF, for these examples, HFCS 42, HFCS 55 and HFCS 90 were identically processed in parallel at a reactor temperature of 200 degrees Celsius, with a reaction/hold time of 7 minutes and with 0.5 percent by weight of sulfuric acid based on the total sugars in the feed, to assess the relationship of the glucose/fructose ratio on product composition and overall sugars accountability for a given set of reaction conditions. The results are shown in FIG. 4.

### EXAMPLES 55-60

In practical terms, it would be preferable for making HMF to be able to use the HFCS product, HFCS 90, with the greatest amount of the more-readily dehydrated fructose. Accordingly, a series of three experiments were conducted in parallel with an HFCS 90 feed at different final dry solids concentrations in the reaction mixture, but otherwise identical conditions of 0.5 weight percent sulfuric acid based on total sugars mass, 200 degrees Celsius reactor temperature with rapid heating of the reaction mixture (40 second feed time) and rapid cooling of the products and a 5 minute time of reaction. The three runs were conducted at 9 percent, 15 percent and 19 percent of final dry solids with the results shown in FIG. 5A. As well, an additional three runs were conducted with these same final dry solids concentrations, but using a reaction time of 7 minutes rather than 5 minutes. These results are shown in FIG. 5B.

### EXAMPLES 61-66

For these examples, an HFCS 90 feed was dehydrated at three different reactor temperatures over both a ten minute reaction/hold time with 10% final dry solids (Examples 61-63) and a seven minute reaction/hold time with 15% final dry solids (Examples 64-66). Analysis of the resultant product mixtures provided the results shown graphically in FIGS. 6A (ten minute runs) and 6b (seven minute runs).

### EXAMPLES 67-94

Using both HFCS 42 and HFCS 90 syrups as feeds, a number of larger-scale continuous runs were conducted at various reaction conditions, using direct steam injection for rapid heating of the feed materials. The apparatus used is shown schematically in FIG. 7, in which a CAT triplex high pressure pump was used to continuously feed a sugars solution into the reactor at a steady rate, as indicated by a micromotion coriolis mass flowmeter and by means of a variable frequency drive. Steam was delivered at a set pressure and injected into the flowing sugars solution to facilitate radial mixing, with steam delivery pressures ranging from 200 psig to 450 psig. Steam flow as adjusted as needed with a flow control valve based on deviations from the desired temperature set point observed at the temperature control element. System back pressures ranged from 140 psig to 440 psig, and reaction setpoint temperatures from 180 degrees Celsius to 210 degrees Celsius. The temperature at the end of the resting tube was recorded and ranged from 95 degrees Celsius to 180 degrees Celsius. The reaction residence time for HFCS 42 solutions were maintained between 5 and 6 minutes, with adjustments to the flowrates being made as necessary to achieve such residence times given the volume of the reactor. The reactor residence time for the HFCS 90 solutions was kept at about 11 minutes. The dry solids concentration of the HFCS 42 solutions was 20 percent by weight, while for the HFCS 90 solutions a dry solids concentration of 10 percent by weight was employed. The results of the larger scale testing are shown in Table 5 below. The reaction product was rapidly cooled for each run (in less than one minute) to 80 degrees Celsius or lower through the use of a brazed plate heat exchanger prior to pressure reduction. In all instances, virtually no insoluble humins were observed to be formed.

TABLE 5
Results of Continuous Larger Scale Testing

| | Reactor | | | Steam | Dry | | | | | |
|---|---|---|---|---|---|---|---|---|---|---|
| | Residence | | System | Delivery | Solids in | | % Molar Yield² | | | |
| Entry | Time | Temp | Pressure | Pressure | Feed | | | Levulinic | C6 | |
| # | (min) | (C )¹ | (psig) | (psig) | (%) | HMF | Furfural | Acid | Sugars | Total |
| 67 | 5.5 | 149 | 320 | 450 | 20 | 16 | 1 | 1 | 60 | 78 |
| 68 | 5.5 | 132 | 308 | 450 | 20 | 10 | 0 | 0 | 69 | 80 |
| 69 | 5.5 | 171 | 310 | 450 | 20 | 22 | 1 | 1 | 51 | 75 |
| 70 | 5.5 | 98 | 430 | 450 | 20 | 3 | 0 | 0 | 82 | 86 |
| 71 | 5.5 | 121 | 430 | 450 | 20 | 12 | 1 | 1 | 70 | 83 |
| 72 | 5.5 | 149 | 430 | 450 | 20 | 22 | 2 | 2 | 54 | 80 |
| 73 | 5.5 | 135 | 440 | 450 | 20 | 16 | 1 | 1 | 63 | 81 |
| 74 | 5.5 | 154 | 211 | 450 | 20 | 11 | 0 | 0 | 73 | 85 |
| 75 | 5.5 | 154 | 210 | 450 | 20 | 11 | 1 | 0 | 67 | 79 |
| 76 | 5.5 | 148 | 208 | 450 | 20 | 9 | 0 | 0 | 75 | 85 |
| 77 | 5.5 | 152 | 213 | 450 | 20 | 11 | 0 | 0 | 71 | 82 |
| 78 | 5.5 | 153 | 210 | 250 | 20 | 8 | 0 | 0 | 79 | 87 |
| 179 | 5.5 | 155 | 220 | 250 | 20 | 6 | 0 | 0 | 83 | 89 |
| 80 | 5.5 | 167 | 210 | 250 | 20 | 14 | 1 | 1 | 71 | 86 |
| 81 | 5.5 | 173 | 210 | 325 | 20 | 12 | 1 | 1 | 74 | 87 |
| 82 | 5.5 | 169 | 208 | 325 | 20 | 21 | 1 | 1 | 61 | 85 |
| 83 | 5.5 | 176 | 220 | 325 | 20 | 19 | 1 | 1 | 65 | 87 |
| 84 | 5.5 | 126 | 240 | 325 | 20 | 22 | 2 | 2 | 57 | 83 |
| 85 | 5.5 | 166 | 217 | 275 | 20 | 14 | 1 | 0 | 78 | 93 |
| 86 | 5.5 | 155 | 215 | 275 | 20 | 16 | 1 | 1 | 76 | 94 |
| 87 | 5.5 | 155 | 218 | 275 | 20 | 20 | 1 | 1 | 70 | 92 |
| 88 | 5.5 | 154 | 224 | 275 | 20 | 16 | 1 | 1 | 73 | 90 |
| 89 | 11 | 119 | 150 | 200 | 10 | 15 | 1 | 0 | 88 | 103 |
| 90 | 11 | 129 | 150 | 200 | 10 | 16 | 1 | 0 | 87 | 104 |
| 91 | 11 | 166 | 150 | 200 | 10 | 26 | 1 | 0 | 69 | 97 |
| 92 | 11 | 175 | 148 | 200 | 10 | 27 | 1 | 1 | 68 | 96 |
| 93 | 11 | 179 | 149 | 200 | 10 | 29 | 1 | 1 | 66 | 96 |
| 94 | 11 | 179 | 149 | 200 | 10 | 31 | 1 | 1 | 64 | 97 |

¹Recorded temperature is the temperature indicated at the end of the reaction resting tube
²Molar yields are calculated from C6 and DP sugars

### EXAMPLE 95

For this example, the apparatus and procedure were used of Examples 1-26, except that in one instance, the room temperature HFCS-42 based sugar solution (6% on a dry solids basis) was fed rapidly into the reactor over the span of two minutes, while in the second run the solution was slowly fed into the reactor over a period of thirty minutes. In each instance, the sugar solutions were then dehydrated over a further sixty (60) minutes in the presence of sulfuric acid (at 0.4 percent by weight based on the total mass of sugars) at a temperature of 170 degrees Celsius. HPLC analysis of the products showed that 96 percent of the sugars could be accounted for with the “rapid feed” method's products, whereas the sugar accountability for the thirty minute feed cycle run was only 43 percent. Combined molar percent yields for the furanic products (HMF, furfural and ethoxymethylfurfural) were 28 percent for the rapid feed method, but only about 16 percent for the thirty minute feed cycle run. The residual sugars were produced at 27 percent molar yield in the rapid feed method, compared to 9 percent for the longer feed cycle.

### EXAMPLES 96 AND 97

The same apparatus and procedure were used as in Example 95, to show the effect of rapid feeding/heating versus more deliberate feeding/heating, for a 22% solution of HFCS-42 (dry solids basis, again) in the synthesis of the HMF ether derivative with ethanol at a 1.1:1 ratio by weight of ethanol:sugar solution to a 12% final dry solids weight. Rather than comparing outcomes of a two minute and a thirty minute feed cycle with a single further reaction time of sixty minutes, however, runs were completed with 5, 7.5, 10, 12.5 and 15 minute reaction times. In addition, the reaction was conducted at 180 degrees, rather than 170 degrees. Results were as reported in Table 6:

TABLE 6

| Gradual Feed/Heat (30 min) | | | Rapid Feed/Heat (2 min) | | | |
|---|---|---|---|---|---|---|
| % | % | % | % | % | % | Reaction |
| selectivity | selectivity | HMF | selectivity | selectivity | HMF | time |
| HMF | furans | yield | HMF | furans | yield | (min) |
| 65 | 80 | 51 | 67 | 74 | 31 | 5 |
| 62 | 82 | 51 | 68 | 76 | 40 | 7.5 |
| 61 | 82 | 50 | 70 | 80 | 47 | 10 |
| 57 | 81 | 49 | 67 | 81 | 50 | 12.5 |
| 47 | 72 | 39 | 67 | 87 | 52 | 15 |

% selectivity of HMF = moles HMF produced/moles sugars reacted * 100.
% selectivity furans = (moles HMF + moles furfural + moles AcMF produced)/moles reacted sugars *100

### EXAMPLES 98 AND 99

The same apparatus and procedure were used as in Examples 96 and 97, except that acetic acid was incorporated rather than ethanol, in the same 1.1:1 ratio by weight, and the sulfuric acid was reduced to 0.2 percent by weight based on the total mass of sugars. In contrast to the results seen with both the synthesis of HMF and the HMF ether with ethanol, however, little advantage was seen with using a rapid feeding/heating cycle as compared to a more gradual feeding/heating cycle. Detailed results are shown in Table 7:

TABLE 7

| Gradual Feed/Heat (30 min) | | | Rapid Feed/Heat (2 min) | | | |
|---|---|---|---|---|---|---|
| % | % | % | % | % | % | Reaction |
| selectivity | selectivity | HMF | selectivity | selectivity | HMF | time |
| HMF | furans | yield | HMF | furans | yield | (min) |
| 45 | 45 | 37 | 41 | 49 | 22 | 5 |
| 48 | 48 | 40 | 39 | 48 | 29 | 7.5 |
| 46 | 46 | 39 | 41 | 51 | 34 | 10 |
| 46 | 46 | 38 | 38 | 48 | 33 | 12.5 |
| 45 | 45 | 37 | 35 | 45 | 32 | 15 |

% selectivity of HMF = moles HMF produced/moles sugars reacted * 100.
% selectivity furans = (moles HMF + moles furfural + moles AcMF produced)/moles reacted sugars *100

What is claimed is:

1. A process for making hydroxymethylfurfural from an aqueous solution including one or more hexoses, comprising subjecting the aqueous hexose solution to an acid-catalyzed dehydration to produce a product mixture including hydroxymethylfurfural and residual unconverted sugars, then separating the product mixture into an hydroxymethylfurfural product and a residual sugars product which is sufficiently free of ethanol fermentation inhibitors to be suitable for use directly as a feed to a fermentation process for producing ethanol.

2. A process according to claim 1, further comprising using residual sugars product directly in an ethanol fermentation, in a fermentation to produce lysine, in a fermentation to produce lactic acid, or as a feed in a process for making a sugar alcohol.

3. A process according to claim 1, further comprising recycling at least a portion of the residual sugars product to make additional hydroxymethylfurfural.

4. A process according to claim 1, wherein the aqueous hexose solution comprises both of glucose and fructose.

5. A process according to claim 4, wherein the glucose and fructose are present in the aqueous hexose solution in the same proportion as in an HFCS 42 corn syrup product, or an HFCS 55 corn syrup product, or an HFCS 90 corn syrup product.

6. A process according to claim 1, wherein the aqueous hexose solution is added to a reactor containing an acid catalyst and which has been preheated substantially to the temperature at which the acid-catalyzed dehydration step is to be conducted.

7. A process according to claim 6, wherein the reaction temperature is from 175 to 205 degrees Celsius.

8. A process according to claim 1, wherein pressurized steam is injected into a reactor containing the aqueous hexose solution and directly heats the aqueous hexose solution to a temperature of from 175 degrees Celsius to 205 degrees Celsius.

9. A process according to any of claims 6-8, wherein the product mixture is rapidly cooled to 50 degrees Celsius and lower in not more than 5 minutes.

10. A process according to any of claims 6-8, wherein the aqueous hexose solution is heated from ambient temperature to the reaction temperature in less than 15 minutes.

11. A process according to claim 1, wherein the dehydration results in a product mixture with from 10 to 55 percent molar yield of hydroxymethylfurfural, from 30 to 80 percent molar yield of residual sugars and not more than 10 percent molar yield of other products.

12. A process according to claim 11, wherein the product mixture comprises from 20 to 55 percent molar yield of hydroxymethylfurfural, from 40 to 70 percent molar yield of residual sugars, and not more than 5 percent molar yield of other products.

13. A process according to claim 1, wherein the product mixture comprises from 40 to 55 percent molar yield of hydroxymethylfurfural, from 25 to 40 percent molar yield of residual sugars, and not more than 5 percent molar yield of other products.

14. A process according to claim 1, wherein separating the product mixture includes one or more iterations of extraction with ethyl acetate to remove hydroxymethylfurfural and other dehydration products from the product mixture.

15. A process according to claim 1, in which the sum of molar yield percentages of hydroxymethylfurfural, residual sugars and levulinic acid in the product mixture exceeds 70 percent.

16. A process according to claim 1, in which the sum of molar yield percentages of hydroxymethylfurfural, residual sugars and levulinic acid in the product mixture exceeds 80 percent.

17. A process according to claim 1, in which the sum of molar yield percentages of hydroxymethylfurfural, residual sugars and levulinic acid in the product mixture exceeds 90 percent.

18. A process for making an hydroxymethylfurfural ether from an aqueous solution including one or more hexoses, comprising subjecting the aqueous hexose solution to an acid-catalyzed dehydration in the presence of an alcohol to produce a product mixture including an hydroxymethylfurfural ether and residual unconverted sugars, then separating the product mixture into an hydroxymethylfurfural ether product and a residual sugars product which is sufficiently free of ethanol fermentation inhibitors to be suitable for use directly as a feed to a fermentation process for producing ethanol.
